(19) United States
(12) Plant Patent Application Publication
(10) Pub. No.: US 2016/0192560 P1
(43) Pub. Date: Jun. 30, 2016

(54) Hops plant named 'Furano 902D go'

(50) ;

(71) Applicant: Sapporo Breweries Limited, Tokyo (JP)

(72) Inventors: Koichiro Koie, Hokkaido (JP); Narushi Suda, Hokkaido (JP); Yutaka Itoga, Hokkaido (JP); Mitsuhiro Uemoto, Hokkaido (JP); Yasunori Arai, Hokkaido (JP); Masanobu Goto, Hokkaido (JP)

(73) Assignee: Sapporo Breweries Limited, Tokyo (JP)

(21) Appl. No.: 14/757,380

(22) Filed: Dec. 21, 2015

(30) Foreign Application Priority Data
Dec. 24, 2014 (JP) 29813

(51) Int. Cl. A01H 5/00

(52) U.S. Cl. PLT/236

(57) ABSTRACT

A new hops plant particularly distinguished by having a cylindrical plant shape, alpha acid content of 13.4% in dry matter, a ratio content of beta to alpha of 0.6, a content of cohumulone to alpha acids of 32%, and a flowery and fruity flavor, is disclosed.

### CROSS REFERENCE TO RELATED APPLICATION

The present application claims priority to Japan Plant Variety Protection Application No. 29813, as filed on Dec. 24, 2014, the entire contents are herein incorporated by reference for all the application teaches and discloses.

### GENUS AND SPECIES

Humulus lupulus L.

### VARIETY DENOMINATION

‘Furano 0902D go’

### BACKGROUND OF THE NEW PLANT

The present invention comprises a new and distinct variety of Hops, botanically known as Humulus lupulus L. and hereinafter referred to by the variety name ‘Furano 0902D go’. ‘Furano 0902D go’ is a selection from a controlled cross-pollination of the proprietary hops female parent ‘990340’ and the proprietary male parent ‘000210’.

The proprietary female hops line ‘990340’ and the proprietary male hops line ‘000210’ were cross-pollinated in Kamifurano, Hokkaido, Japan and seeds were obtained. The seeds were sown and plants were grown for evaluation. A plant line was selected in March of 2009 in Kamifurano, Hokkaido, Japan and named ‘Furano 0902D go’. In 2009, ‘Furano 0902D go’ was first vegetatively propagated by in Kamifurano, Hokkaido, Japan via vegetative cuttings. ‘Furano 0902D go’ was found to reproduce true to type in successive generations of asexual propagation via vegetative cuttings in Kamifurano, Hokkaido, Japan.

### SUMMARY

The following are the most outstanding and distinguishing characteristics of this new variety when grown under normal horticultural practices in Kamifurano, Hokkaido, Japan.

1. Cylindrical plant shape;

2. Alpha acid content of 13.4% in dry matter;

3. Ratio content of beta to alpha of 0.6;

4. Content of cohumulone to alpha acids of 32%; and

5. A flowery and fruity flavor.

### DESCRIPTION OF THE PHOTOGRAPHS

This hops plant is illustrated by the accompanying photographs which show the plant's overall plant shape, leaf shape and cone shape. The photographs are of six-year-old plants grown in Kamifurano, Hokkaido, Japan in August 2014. The colors shown are as true as can be reasonably obtained by conventional photographic procedures.

FIG. 1 shows the plant shape of ‘Furano 0902D go’.

FIG. 2 shows the leaf shape of ‘Furano 0902D go’ and three commercial hops varieties in Japan. The lower right shows ‘Furano 0902D go’, the upper right shows ‘Little Star’, the upper left shows ‘Shinsyu Wase’, and the lower left shows ‘Furano Special’.

FIG. 3 shows the cone shape of ‘Furano 0902D go’.

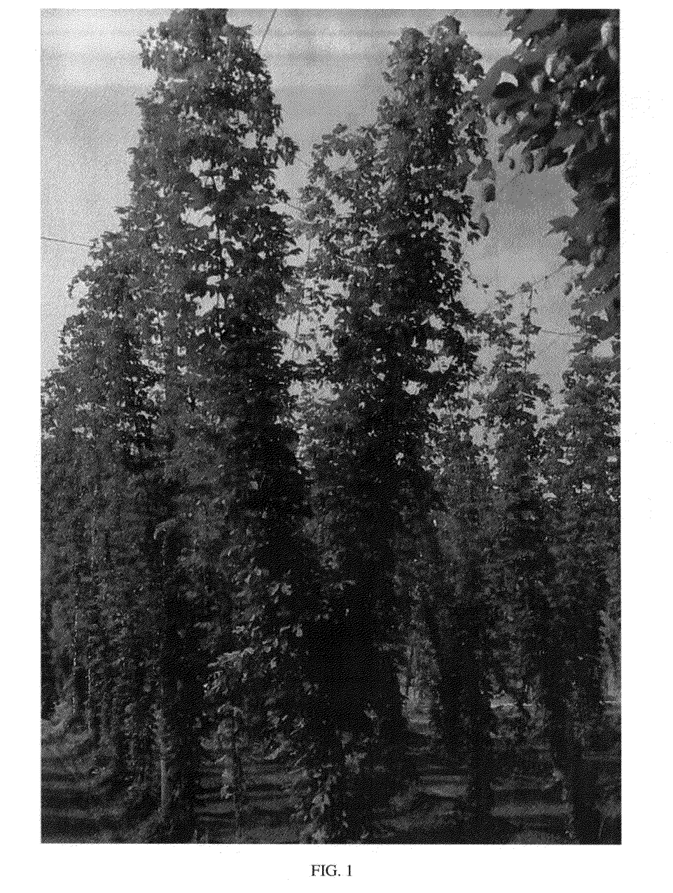

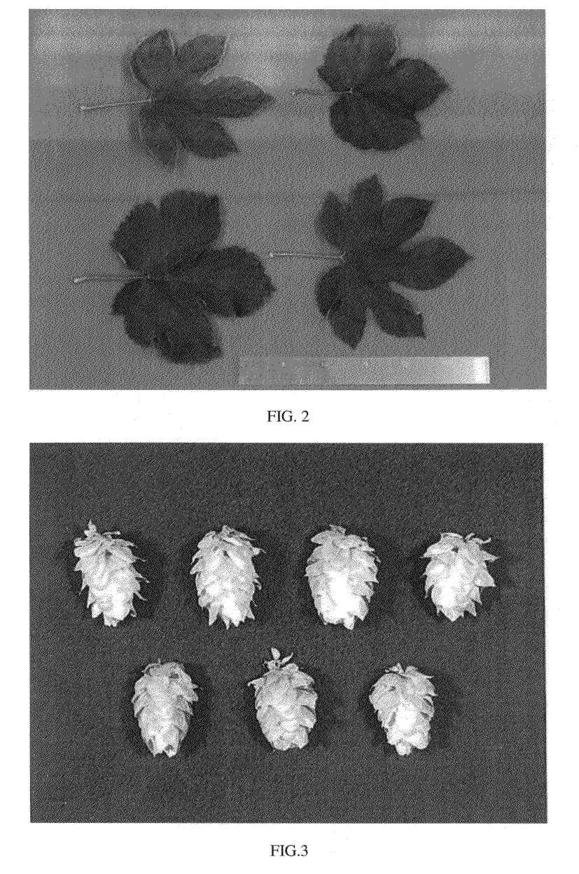

### DESCRIPTION OF THE NEW VARIETY

The following detailed descriptions set forth the distinctive characteristics of ‘Furano 0902D go’. The data, which define these characteristics, were collected from asexual reproductions carried out in Kamifurano, Hokkaido, Japan. Data was collected on five- and six-year-old plants in Kamifurano, Hokkaido, Japan during the 2013 and 2014 growing season. Color references are to Japan Horticultural Plant Color Chart (1987, Japan Color Research Institute).
Classification:
   Family.—Cannabaceae.
   Classification.—Humulus lupulus L.
   Common name.—Hops.
Propagation:
   Type.—Vegetative cuttings.
Plant description:
   Plant and growth habit.—The plant growth type is normal, not dwarf.
   Plant shape.—Cylindrical.
Lateral branch description:
   Length.—Average is 0.82 m.
Foliage description:
   Length.—Average is 13.8 cm.
   Color.—Dark Green (Japan Color Chart No. 3707).
   The number of lobes.—Three to five.
Inflorescence buds:
   Cone size.—Medium.
   Cone shape.—Medium, ovate
   A hundred cone weight.—Light (15.2 g in dry matter).
   Color.—Vivid yellow green (Japan Color Chart No. 3311).
   Flowering date (The date when half of the plants being in bloom).—July 16^{th}.
   Harvest date.—August 30th.
Pedicels: Not observed
Reproductive organs:
   Stamens.—Not observed.
   Pistils.—Not observed.
Disease and pest/insect resistance: None observed
Brewing characteristics: Content of alpha acid is 13.4% in dry matter. Ratio content of beta to alpha is 0.6. Content of cohumulone to alpha acids is 32%. Flavor of ‘Furano 0902D go’ is flowery and fruity.

### COMPARISON WITH PARENTAL LINES AND KNOWN VARIETY

‘Furano 0902D go’ is a distinct variety of hops. ‘Furano 0902D go’ is distinguished from its female parent ‘990340’ as described in Table 1:

TABLE 1
Comparison with Parental Lines

| | Variety | Female parent |
|---|---|---|
| Characteristic | ‘Furano 0902D go’ | ‘990340’ |
| Flowering date | 16^{th} of July | 15th of July |
| Harvest date | 30^{th} of August | 29th of August |
| Content of alpha acid in dry | 13.4% | 11.3% |
| matter | | |
| Content of cohumulone in | 32% | 25% |
| alpha acid | | |

‘Furano 0902D go’ is distinguished from the commercial hops variety ‘Little Star’ (not patented). Differences between the two varieties are described in Table 2:

TABLE 2
Comparison with Similar Variety

| | Variety | Commercial line |
|---|---|---|
| Characteristic | ‘Furano 0902D go’ | ‘Little Star’ |
| Flowering date | 16th of July | 7th of July |
| Harvest date | 30th August | 21st August |
| Content of alpha acid in | 13.4% | 7.3% |
| dry matter | | |
| Ratio content of beta | 0.6 | 0.7 |
| acid to alpha acid | | |
| Content of cohumulone | 32% | 22% |
| in alpha acid | | |

We claim:

1. A new and distinct variety of hops plant named ‘Furano 0902D go’ as illustrated and described herein.
